(19) United States
(12) Patent Application Publication
(10) Pub. No.: US 2025/0195197 A1
(43) Pub. Date: Jun. 19, 2025

(54) DECELLULARIZED NERVE CONDUIT PREPARED USING SUPERCRITICAL FLUID EXTRACTION PROCESS AND USES THEREOF

(71) Applicants: DOF INC., Gyeonggi-do, Hwaseong-si (KR); SEOUL NATIONAL UNIVERSITY HOSPITAL, Seoul (KR)

(72) Inventors: Yong Woo SHIN, Gyeonggi-do, Hwaseong-si (KR); Seong-Rae NOH, Gyeonggi-do, Hwaseong-si (KR); Chan Young HEO, Gyeonggi-do, Yongin-si (KR); Sun Young NAM, Gyeonggi-do, Anyang-si (KR)

(21) Appl. No.: 18/836,263

(22) PCT Filed: Dec. 30, 2022
(86) PCT No.: PCT/KR2022/021695

§ 371 Date: Aug. 6, 2024

(30) Foreign Application Priority Data
Feb. 8, 2022 (KR) 10-2022-0016319

(51) Int. Cl. A61F 2/04; A61L 27/36

(52) U.S. Cl. CPC A61F 2/04; A61L 27/3604; A61L 27/3675; A61L 27/3687

(57) ABSTRACT

The present invention relates to a decellularized nerve conduit prepared using a supercritical fluid extraction process. More specifically, the present invention relates to a decellularized nerve conduit having optimized mechanical properties such as tensile strength and elastic restoring force by using a supercritical fluid extraction process. The nerve conduit according to the present invention is decellularized without a surfactant treatment, so that not only are mechanical properties maintained, but there is no toxicity due to residual surfactant, and transplant rejection is minimized, so that the nerve conduit can be usefully employed in treating patients with nerve defects.

### TECHNICAL FIELD

The present invention relates to a decellularized nerve conduit prepared using a supercritical fluid extraction process. More specifically, the present invention relates to a decellularized nerve conduit with optimized mechanical properties such as tensile strength and elastic recovery force using a supercritical fluid extraction process.

### BACKGROUND ART

Nerve defects caused by various accidents, diseases, cancer surgeries, etc. occur at all ages. Natural regeneration is almost impossible for central nervous system damage, such as brain damage or spinal cord damage, and if peripheral nerves are damaged, there is a possibility that they may regenerate over time, but full functional recovery is difficult.

Various methods for treating these nerve defects include nerve stretching, autologous nerve conduit transplantation, and artificial nerve conduit transplantation, but the treatments have side effects. The nerve stretching has a high risk of nerve breaking during nerve stretching, and in the case of breaking, regeneration is impossible. In the case of autologous nerve conduit transplantation, surgery is required on both a damaged area and a transplanted area, and there is a limitation in that a nerve in an area from which a nerve conduit was removed loses function thereof. Additionally, a calf nerve used in autologous nerve conduit transplantation may not be transplanted for diabetic patients. In the case of artificial nerve conduit transplantation, a hollow transplanted nerve grows without direction; therefore, the regeneration success rate is lower than that of autologous nerve conduit transplantation, removal should be ensured when necessary, and the cost is expensive.

Specifically, a technology has been developed to decellularize donated neural tubes by treating the same with surfactants, but there are limitations such as denaturation of proteins such as collagen and destruction of growth factors due to treatment with surfactants. Additionally, it is not easy to remove the remaining surfactant, and if the surfactant is not completely removed, the nerve tubes may be toxic. In particular, surfactants make it difficult to maintain structural and histological forms such as the tubular shape of a neural tube, mechanical properties such as tensile strength and elasticity deteriorate, making suturing difficult during surgery for connecting a nerve conduit, and if a surfactant remains in the nerve conduit, there is a difficulty because transplant rejection is caused due to toxic substances (U.S. Pat. No. 7,402,319).

Meanwhile, there is also a technology to fill a conduit with a decellularized extracellular matrix of animal nervous tissue to replace tissue decellularization using a surfactant (Korean Patent No. 10-1608618), but this has a complicated production process, such as collecting animal nervous tissue, decellularizing the tissue, and filling into a gel-shaped conduit, and has a problem of using a separate artificial conduit.

### DISCLOSURE OF THE INVENTION

### Technical Problem

Accordingly, the present inventors conducted research to develop a nerve conduit transplant material that remedies the problems of transplant rejection and cost, and at the same time remedies the problem in deterioration of mechanical properties caused by decellularization by chemical processing methods. As a result, the present invention was completed by establishing an optimized supercritical fluid treatment process without surfactant treatment and obtaining a decellularized nerve conduit with improved mechanical properties.

### Technical Solution

To this end, an aspect of the present invention provides a decellularized nerve conduit, wherein the nerve conduit, which is separated from a subject, has tensile strength of 2 to 5 N, Young's modulus of 20 to 80 Mpa, and elastic restoring force of 30 to 80% compared to native tissue.

Another aspect of the present invention provides a production method of a decellularized nerve conduit, the production method including: a) pretreating nervous tissue separated from a subject with a hypertonic buffer; b) extracting the pretreated nervous tissue with supercritical fluid; and c) washing, with a phosphate buffer, the nervous tissue extracted with supercritical fluid.

### Advantageous Effects

A nerve conduit according to the present invention is decellularized without surfactant treatment and maintains mechanical properties such as tensile strength and elasticity, so that the nerve conduit is easy for transplant surgery and suturing and there is no short circuiting again after connection surgery. In addition, unlike a conventional artificial nerve conduit, there is no need for separate removal surgery after surgery, and there is no toxicity due to residual surfactant, so that the nerve conduit is suitable for insertion or implantation in patients with nerve defects. Therefore, the nerve conduit of the present invention obtained through an optimized supercritical fluid treatment process is free of toxicity and transplant rejection, and is usefully used in the treatment of patients with nerve defects.

### BRIEF DESCRIPTION OF THE DRAWINGS

FIG. 1 is a photograph showing a nerve conduit obtained after a supercritical fluid extraction process according to an embodiment of the present invention.

FIG. 2 is a photograph of measuring tensile force and elasticity of a nerve conduit obtained after a supercritical fluid extraction process according to an embodiment of the present invention using a universal tensile machine (UTM).

FIG. 3 is a graph showing the results of measuring tensile strength of a nerve conduit obtained after a supercritical fluid extraction process according to an embodiment of the present invention. Here, Thick is a thick pig nervous tissue sample with a size of 5 mm (D)×10 mm (L), and Thin is a thin pig nervous tissue sample with a size of 2.5 mm (D)×10 mm (L). In addition, Native (N) is an untreated group and indicates native tissue, Supercritical process (SC) is a test group decellularized by treatment with supercritical fluid, and Detergent is a positive control decellularized by treatment with a surfactant.

FIG. 4 is a graph showing the results of measuring Young's modulus of a nerve conduit obtained after a supercritical fluid extraction process according to an embodiment of the present invention. In addition, Native (N) is an untreated group and indicates native tissue, Supercritical process (SC) is a test group decellularized by treatment with supercritical fluid, and Detergent is a positive control decellularized by treatment with a surfactant.

FIG. 5 is a photograph of measuring tissue strength and resilience using UTM equipment for a nerve conduit obtained after a supercritical fluid extraction process according to an embodiment of the present invention.

FIGS. 6A and 6B show raw data obtained by measuring tissue strength using UTM equipment for a nerve conduit without performing a supercritical fluid extraction process and a decellularized nerve conduit after a supercritical fluid extraction process, respectively, according to an embodiment of the present invention. Specifically, this is a graph showing the results of measurements from 1 to 10 cycles in a compression mode at a speed of 0.03 mm/min. The graph depicts changes in load values at the starting and ending points measured during repeated movements of the probe.

FIG. 7 shows a graph obtained by measuring tissue strength of a nerve conduit without performing a supercritical fluid extraction process and a decellularized nerve conduit after a supercritical fluid extraction process, respectively, using UTM equipment, and by overlapping raw data thereof, according to an embodiment of the present invention.

FIG. 8 is a bar graph showing tissue strength measured in 10 cycles using UTM equipment for a nerve conduit without performing a supercritical fluid extraction process and a decellularized nerve conduit after a supercritical fluid extraction process, respectively, according to an embodiment of the present invention.

FIGS. 9A and 9B show measurements of the degrees of resilience of a nerve conduit without performing a supercritical fluid extraction process and a decellularized nerve conduit after a supercritical fluid extraction process, respectively, using UTM equipment for 1 to 6 cycles, according to an embodiment of the present invention. FIG. 9A shows a difference value between a starting point and end point of measuring the resilience of a nerve conduit calculated for each cycle, and FIG. 9B shows a relative value between samples calculated for each cycle using the derived difference value.

FIG. 10A is an 1% agarose gel electrophoresis image confirming the remaining amount of DNA of a nerve conduit without a supercritical fluid extraction process (N1, N2), a decellularized nerve conduit after a supercritical fluid extraction process (SC1, SC2), and a nerve conduit treated with a surfactant (D1, D2) according to an embodiment of the present invention. At this time, N1 and N2, SC1 and SC2, and D1 and D2 are loaded with DNA obtained from two different parts of the same tissue.

FIG. 10B is a diagram showing the results of extracting DNA from a nerve conduit obtained after a supercritical fluid extraction process by a salting out method and performing quantitative analysis of the remaining DNA according to an embodiment of the present invention. Here, Native (N) is an untreated group and indicates native tissue, Supercritical process (SC) is a test group decellularized by treatment with supercritical fluid, and Detergent is a positive control decellularized by treatment with a surfactant.

FIG. 11 is a photograph showing Hematoxylin & Eosin (H&E) and DAPI staining results of a nerve conduit obtained after a supercritical fluid extraction process according to an embodiment of the present invention. Herein, purple is a counterstain to contrast the cell nuclear area, and red is a counterstain to contrast the cytoplasm or extracellular structures. Blue is DNA stained using DAPI fluorescence staining. The black bar represents a size of 100 μm.

FIG. 12 is a graph showing quantitative analysis of a collagen content based on dry weight of a nerve conduit obtained after a supercritical fluid extraction process according to an embodiment of the present invention.

FIG. 13 is a graph showing quantitative analysis of an elastin content based on dry weight of a nerve conduit obtained after a supercritical fluid extraction process according to an embodiment of the present invention.

FIG. 14 shows the results of Western blot analysis confirming the degree of laminin preservation in a nerve conduit obtained after a supercritical fluid extraction process according to an embodiment of the present invention.

FIG. 15 is a diagram confirming whether DNA remains in a nerve conduit obtained after a supercritical fluid extraction process according to an embodiment of the present invention. DNA was extracted using the DNeasy blood and tissue kit (Cat. no. 69506, Qiagen, Germany) from a nerve conduit (Sc-CO₂ nerve) decellularized by supercritical fluid treatment compared to native pig nervous tissue (Native), and quantitative results (unit: ng/mg) of double stranded DNA (dsDNA) remaining measured using (A) Nanodrop and (B) Qubit and a (C) DNA electrophoresis image are shown.

FIG. 16 is a microscope photograph taken at different magnifications of Hematoxylin & Eosin (H&E) staining results of a nerve conduit obtained after a supercritical fluid extraction process according to an embodiment of the present invention. The extent of removal of cell nuclei (purple staining) of a decellularized nerve conduit by supercritical fluid treatment was confirmed compared to pig nervous tissue. Here, Native indicates pig nervous tissue (A, B, and C) as the untreated group, and Sc-CO₂ nerve indicates test groups (D, E, and F) that were decellularized by treatment with supercritical fluid.

FIG. 17 is a micrograph taken at different magnifications of DAPI staining results of a nerve conduit obtained after a supercritical fluid extraction process according to an embodiment of the present invention. The extent of removal of cell nuclei (blue staining) of a decellularized nerve conduit by supercritical fluid treatment was confirmed compared to pig nervous tissue. Herein, Native indicates the untreated group of pig nervous tissue (A and B), and Sc-CO₂ nerve indicates the test group decellularized by treatment with supercritical fluid (C and D).

FIG. 18 is a diagram confirming whether an extracellular matrix (ECM) of a nerve conduit obtained after a supercritical fluid extraction process according to an embodiment of the present invention is preserved. Microphotographs (A to D) confirming the preservation of collagen (blue staining) through Masson's Trichrome staining of a nerve conduit decellularized by supercritical fluid treatment compared to pig nervous tissue, and a quantitative graph of collagen (E) and hyaluronic acid (F) measured using a commercially available kit are shown. Here, Native indicates pig nervous tissue as an untreated group, and Sc-CO₂ nerve refers to a test group decellularized by treatment with supercritical fluid.

FIG. 19 is a diagram confirming the cytotoxicity of a nerve conduit obtained after a supercritical fluid extraction process according to an embodiment of the present invention. A graph (A) quantifying the survival rate of fibroblasts (NIH-3T3 fibroblasts) cultured on nerve conduit tissue decellularized by supercritical fluid treatment using an MTT method, and a micrograph (B) showing the migration of fibroblasts, of which nuclei were stained (blue) using H&E staining, into nerve conduit tissue are shown.

FIG. 20 is a diagram confirming a nerve regeneration effect on a nerve defect rat model of a nerve conduit obtained after a supercritical fluid extraction process according to an embodiment of the present invention. This image shows a regenerated nervous tissue area through H&E and DAPI staining 6 months after nerve conduit transplantation in a rat model with a 10 mm sciatic nerve defect. Herein, A and D represent the nervous tissue of a normal rat, and B and E represent the naturally regenerated nervous tissue of a neural defect model rat after 6 months as a negative control. C and F show regenerated nervous tissue 6 months after transplantation of a nerve conduit obtained after a supercritical fluid extraction process in a nerve defect model rat.

FIG. 21 is a diagram confirming the growth of Schwann cells after transplantation of a nerve conduit into a rat model of nerve defects obtained after a supercritical fluid extraction process according to an embodiment of the present invention. An image (top) confirming a regenerated nervous tissue area through S100 fluorescent immunostaining and DAPI staining 6 months after nerve conduit transplantation in a rat model of nerve defect and a graph (bottom) quantifying the same are shown. Here, Normal represents nervous tissue of a normal rat, and Control (-) represents naturally regenerated nervous tissue of a neural defect model rat after 6 months as a negative control. Sc-CO₂ nerve represents the regenerated nervous tissue 6 months after transplantation of a nerve conduit obtained after a supercritical fluid extraction process in a nerve defect model rat. Additionally, * is p-value <0.05, and ** indicates p-value <0.01. The scale bar on each image represents 50 μm.

FIG. 22 is a diagram showing the results of a walking track analysis after transplantation of a nerve conduit in nerve defect rat model obtained after a supercritical fluid extraction process according to an embodiment of the present invention. The gait trajectory analysis results of group (A) that naturally regenerated 6 months after nerve defect and group (B) that regenerated 6 months after nerve conduit transplantation obtained after a supercritical fluid extraction process as negative controls, and the results of quantitative analysis of Sciatic Functional Index (SFI) (bottom of C) based on a photo (top of C) of the walking path of a nerve defect rat are shown. Here, *** is p-value <0.001.

### BEST MODE FOR CARRYING OUT THE INVENTION

### Decellularized Nerve Conduit

An aspect of the present invention provides a decellularized nerve conduit separated from a subject, the nerve conduit having the following characteristics:

The nerve conduit has tensile strength of 2 to 5 N, Young's modulus of 20 to 80 Mpa, and elastic restoring force of 30 to 80% compared to native tissue.

As used herein, the term “decellularized nerve conduit (nerve conduit)”, which refers to a connector that connects both ends of a defective nerve and serves as a guide for nerve regeneration, fixes both ends of the severed nerve within a nerve conduit and guides the connection of the nerve into the conduit. By using a decellularized nerve conduit, it is possible to prevent the infiltration of scar tissue that interferes with nerve regeneration and guide nerve regeneration in the correct direction. In addition, the decellularized nerve conduit provides the advantage of maintaining nerve regeneration-promoting substances secreted by the nerve itself within the conduit and preventing substances that interfere with regeneration from entering the conduit. The nerve conduit provides a controlled microenvironment, and trophic factors secreted from damaged nerves are concentrate within the conduit to promote axon growth.

The nerve conduit must have mechanical properties enabling to maintain the space inside the conduit while nerve regeneration occurs. The nerve conduit must have appropriate elasticity and tensile strength so that the distal part of the nerve conduit remains stable despite movement of the treatment area after insertion of the nerve conduit. The nerve conduit must be made of a material that does not damage normal tissues around the treatment area and must be easy to operate.

As used herein, the term “decellularization” is a new method of producing an artificial scaffold by removing cells from an entire organ while maintaining the original structure of the target transplanted tissue or organ. In a decellularization process, cellular components are removed from tissue, but an extracellular matrix and some growth factor proteins are preserved. Therefore, various extracellular matrix components, including collagen, fibronectin, and laminin, preserved in decellularized tissue provide a three-dimensional microenvironment similar to that in intact tissue, and thus it may improve the survival, proliferation and differentiation of cultured cells.

In the present invention, decellularization may be performed by supercritical fluid extraction without surfactant treatment, but is not limited thereto.

As used herein, the term “supercritical fluid extraction” or “supercritical extraction” is a method of separating substances using a supercritical fluid, which has characteristics intermediate between gas and liquid, existing above the critical point, that is, critical temperature and critical pressure. The supercritical fluid extraction uses a combination of a solvent extraction principle in which soluble components contained in raw materials are dissolved into supercritical fluid due to the difference in solubility between the extracted raw materials and the supercritical fluid and a distillation principle that is an evaporation phenomenon in which solute molecules contained in raw materials transition from a high-density condensed phase to supercritical fluid of a low-density expanded phase.

As used herein, the term “supercritical fluid” refers to something that is in a gaseous state under normal conditions but is a fluid above the critical temperature and critical pressure. Supercritical fluid suitable for use in the present invention is not particularly limited, but include, for example, carbon dioxide, nitrogen, nitrous oxide, methane, ethylene, propane, and propylene. Preferably, carbon dioxide with a critical temperature of 31° C. and a critical pressure of 72.8 atm may be used.

In the present invention, decellularization may be performed by adding a “co-solvent” in addition to the supercritical fluid when extracting the supercritical fluid. The co-solvent may be added for the purpose of increasing the extractability and solubility of the supercritical fluid, and ethanol, methanol, petroleum ether, acetonitrile, hexane, etc. may be used as the co-solvent, although not limited thereto. At this time, the co-solvent may preferably be ethanol.

The decellularized nerve conduit of the present invention may be derived from nervous tissue separated from the subject. The subject may be a subject belonging to the same or different species as the subject to which the nerve conduit is to be transplanted or inserted, and specifically, may be mammals including a human, mouse, rat, monkey, chimpanzee, orangutan, horse, cow, pig, cat, dog, and rabbit, but is not limited thereto.

When the decellularized nerve conduit of the present invention is compared with the native tissue separated from the above subject, that is, compared with the tissue without decellularization, 90% or more, 91% or more, 92% or more, 93% or more, 94% or more, 95% or more, 96% or more, 97% or more, 98% or more, 99% or more, or 100% of the cells may be removed.

As used herein, the term “tensile strength” refers to the maximum stress until a sample piece is broken by a tensile load, and means the maximum load until rupture divided by an original cross-sectional area of a test specimen. The greater the tensile strength is, the greater the force is required to fracture the support, and this may also mean that the nerve conduit maintains its shape well not only when applied to the nerve defect but also during distribution and storage.

The tensile strength measured in the present invention may be measured using a universal tensile machine. Additionally, in the present invention, the tensile strength of the nerve conduit may be wet tensile strength measured under wet conditions, that is, in a wet state in sterile distilled water, but is not limited thereto.

In the present invention, the decellularized nerve conduit may have a tensile strength of 2 to 5 N, 2.2 to 4.8 N, 2.4 to 4.6 N, 2.6 to 4.4 N, 2.8 to 4.2 N, or 3.0 to 4.0 N under wet conditions based on the nerve conduit specimen having a diameter of 2 mm to 6 mm and a length of 8 mm to 12 mm. At this time, the nerve conduit sample may preferably have a tensile strength of 2 to 4 N, or 2.5 to 3.5 N, based on a diameter of 2 mm to 4 mm and a length of 8 mm to 12 mm.

As used herein, the term “Young's modulus” also referred to as “elastic modulus” or “elastic modulus”, refers to the ratio of the pressure (stress) of an object to the deformation of the object. Young's modulus is the elastic modulus that indicates the degree to which an object deforms when pressure is applied. The basic principle of Young's modulus measurement is that an object undergoes elastic deformation when compressed or expanded, and returns to its original shape when the load is removed. For example, more deformation occurs in a flexible object compared to a stiff object, and a high Young's modulus value means that an object is inelastic or stiff.

Young's modulus measured in the present invention may be measured using a universal tensile machine. Additionally, in the present invention, the Young's modulus of the nerve conduit is not limited thereto, but may be measured under wet conditions.

In the present invention, the decellularized nerve conduit may have Young's modulus of 20 to 80 MPa, 21 to 75 MPa, 22 to 70 MPa, 23 to 68 MPa, 24 to 66 MPa, 25 to 64 MPa, 26 to 63 MPa, 27 to 63 MPa, or 27 to 62 Mpa under wet conditions based on the nerve conduit specimen having a diameter of 2 mm to 6 mm and a length of 8 mm to 12 mm. At this time, preferably, based on the nerve conduit sample having a diameter of 2 mm to 4 mm and a length of 8 mm to 12 mm, the Young's modulus may be 25.2 to 80 MPa, 25.4 to 75 MPa, 25.6 to 70 MPa, 25.8 to 68 MPa, 26.0 to 66 MPa, 26.2 to 65 MPa, 26.4 to 64 MPa, 26.6 to 63 MPa, 26.8 to 62.8 MPa, 27.0 to 62.6 MPa, 27.2 to 62.4 MPa, 27.4 to 62.2 MPa, or 27.6 to 62.0 MPa.

As used herein, the term “elastic restoring force” means the ratio of the degree to which deformation of the object caused by the compressive load is induced and the degree to which the induced deformation is restored to its original state when a compressive load is applied to the object with a set number of repetitions.

The elastic restoring force measured in the present invention may be measured using a universal tensile machine. Additionally, in the present invention, the elastic restoring force of the nerve conduit is not limited thereto, but may be measured under wet conditions.

Additionally, the elastic restoring force of the nerve conduit may be measured by applying a compressive load for 1 to 10 cycles, 2 to 9 cycles, 3 to 8 cycles, 4 to 7 cycles, or 5 to 6 cycles, although not limited thereto. At this time, the compressive load is not limited thereto, but may be applied at a rate of 0.01 to 0.05 mm/min, 0.02 to 0.04 mm/min, or 0.03 mm/min.

In the present invention, the decellularized nerve conduit may exhibit an elastic restoring force of 30 to 80% or 40 to 60% of that of the native tissue under wet conditions, but is not limited thereto.

A nerve conduit according to the present invention is not limited thereto, but may have any one characteristic selected from the following group based on dry weight:
a DNA residual amount of 15 to 50 ng/mg;
a collagen content of 300 to 1000 μg/mg; and
an elastin content of 10 to 60 μg/mg.

In the present invention, the nerve conduit may contain a residual amount of DNA of 50 ng/mg or less based on dry weight. Preferably, 15 to 50 ng/mg, 16 to 45 ng/mg, 17 to 40 ng/mg, 18 to 35 ng/mg, 19 to 30 ng/mg, or 20 to 25 ng/mg may be contained, but is not limited thereto.

In the present invention, the nerve conduit may contain a collagen content of 300 μg/mg or more based on dry weight. Preferably, 300 to 1000 μg/mg, 400 to 980 μg/mg, 500 to 960 μg/mg, 600 to 940 μg/mg, 700 to 920 μg/mg, 800 to 900 μg/mg, or 850 to 900 μg/mg may be contained, but is not limited thereto.

In the present invention, the nerve conduit may contain an elastin content of 10 μg/mg or more based on dry weight. Preferably, 10 to 60 μg/mg, 20 to 59 μg/mg, 30 to 58 μg/mg, 40 to 57 μg/mg, 50 to 56 μg/mg, or 52 to 55 μg/mg may be contained, but is not limited thereto.

A nerve conduit according to the present invention is not limited thereto, but may have any one characteristic selected from the following group compared to native tissue:
a DNA remaining amount of 2 to 6%;
a collagen content of 50 to 160%;
an elastin content of 20 to 120%;
a laminin content of 40 to 95%;
tensile strength of 90 to 110%;
Young's modulus of 80 to 110%; and
elastic restoring force of 30 to 80%.

In the present invention, the nerve conduit may contain 6% or less of DNA residual compared to native tissue, that is, compared to the nerve conduit before decellularization or without decellularization. Preferably, 2 to 6%, 2 to 5%, 2 to 4%, or 2 to 3% may be contained but is not limited thereto.

That is, the nerve conduit may have had more than 94% of its cells removed compared to the native tissue. Preferably, 94 to 98%, 95 to 98%, 96 to 98%, or 97 to 98% may be removed, but is not limited thereto.

The nerve conduit according to the present invention may have extracellular matrix components, such as collagen, elastin, and laminin, maintained at a predetermined content compared to the native tissue.

Specifically, in the present invention, the nerve conduit may contain 50% or more of a collagen content compared to the native tissue. Preferably, 50 to 160%, 60 to 155%, 70 to 150%, 80 to 145%, 90 to 140%, 100 to 140%, or 110 to 140% may be contained, but is not limited thereto.

In the present invention, the nerve conduit may contain 20% or more of elastin content compared to the native tissue. Preferably, 20 to 120%, 30 to 118%, 40 to 116%, 50 to 114%, 60 to 113%, 70 to 112%, 80 to 111%, or 90 to 110% may be contained, but is not limited thereto.

In the present invention, the nerve conduit may contain a laminin content of 40% or more compared to the native tissue. Preferably, 40 to 95%, 60 to 94%, 80 to 93%, 85 to 92%, or 90 to 91% may be contained, but is not limited thereto.

In the present invention, the nerve conduit may have tensile strength of 90 to 110%, 92 to 109%, 94 to 108%, or 96 to 107% of the native tissue, but is not limited thereto.

In the present invention, the nerve conduit may have Young's modulus of 80 to 110%, 81 to 109%, 82 to 108%, or 83 to 107% compared to the native tissue, but is not limited thereto.

In the present invention, the nerve conduit may have an elastic restoring force of 30 to 80%, or 40 to 60% of the native tissue, but is not limited thereto.

In exemplary implementations of the present invention, a nerve conduit does not contain a surfactant. In the case of a nerve conduit decellularized by conventional surfactant treatment, there were problems such as denaturation of proteins such as collagen due to surfactant treatment, a subsequent decrease in the density of neural tube tissue, or destruction of growth factors. In addition, it was not easy to remove the remaining surfactant, and there was a problem that there might be toxicity if the surfactant was not completely removed.

In addition, mechanical properties such as tensile strength and elasticity of the nerve conduit deteriorate due to surfactants; therefore, when performing surgery to connect the nerve conduit, suturing is difficult; and if a surfactant remained in the nerve conduit, transplant rejection was caused due to toxic substances.

However, the decellularized nerve conduit obtained according to the supercritical fluid process according to the present invention is decellularized to the same level as a decellularized nerve conduit treated with a conventional surfactant, and has the characteristics of maintaining histological form and mechanical properties. In particular, nervous tissue decellularized according to the supercritical fluid process has the advantage of significantly superior preservation of extracellular matrix (ECM) such as collagen, elastin, and laminin within the nerve conduit compared to nerve conduit treated with surfactant.

Therefore, the nerve conduit according to the present invention is decellularized without a loss of an extracellular matrix while maintaining histological form and mechanical properties, so as to be usable as a transplant material.

In one embodiment of the present invention, the decellularized nerve conduit with the above advantages was transplanted into a rat model of sciatic nerve damage, and then the sciatic nerve functional index (SFI) was analyzed to evaluate its effectiveness. An SFI index of 0 indicates complete normality, and an index of −100 indicates that the sciatic nerve has been completely severed. As a result of the analysis, it was found that the SFI index was higher when the nerve conduit was transplanted decellularized by the supercritical process compared to the control that was naturally regenerated without nerve conduit transplantation (FIG. 22). Therefore, it is possible to know that when the decellularized nerve conduit is transplanted, the nerve regeneration and motor function recovery effects are superior compared to the control.

In the present invention, the SFI index derived by transplantation of the nerve conduit is not limited thereto, but may be −60 or more, −55 or more, −50 or more, −45 or more, or −40 or more. Preferably, the index may be −40 or more.

A decellularized nerve conduit according to the present invention, which has the above-mentioned series of characteristics, has an excellent motor function recovery effect when applied in vivo to a nerve cut model, and may be usable as a useful graft material for repairing damaged nerves.

### Production of Decellularized Nerve Conduit

The decellularized nerve conduit according to the present invention may be produced by a production method including extracting nervous tissue separated from a subject with a supercritical fluid.

The terms “decellularization,” “subject,” “nerve conduit,” and “supercritical fluid” are as described above.

In the present invention, the supercritical fluid may produce a decellularized nerve conduit by extracting and decellularizing lipid components in the nervous tissue separated from the subject based on solubility, specifically phospholipid components, which are the main component of a cell membrane.

The supercritical fluid may be selected from the group consisting of carbon dioxide gas, ammonia gas, nitrogen gas, nitrogen monoxide (NO) gas, nitrogen dioxide (NO₂) gas, nitrous oxide (N₂O) gas, sulfur dioxide gas, hydrogen gas, water vapor, saturated hydrocarbons, unsaturated hydrocarbons, aromatic compounds and mixed gas thereof. Specifically, the supercritical fluid may be carbon dioxide gas, but the type of supercritical fluid is not limited thereto as long as it is possible to efficiently produce a nerve conduit by removing most of the cells of the nervous tissue while maintaining the structural shape as well as the mechanical properties of the nervous tissue. When using carbon dioxide gas as the supercritical fluid, carbon dioxide has a low critical temperature (31° C.) and critical pressure (73 bar), so that the condition may be easily adjusted to supercritical conditions, and carbon dioxide exists widely in the natural world and has the advantage of being colorless, odorless, harmless to the human body, and chemically stable.

In the present invention, the supercritical extraction may be performed under pressure conditions of 0 to 1000 bar, 30 to 900 bar, 60 to 800 bar, 90 to 700 bar, 120 to 600 bar, 150 to 500 bar, or 200 to 400 bar.

Specifically, the pressure of the supercritical extraction is not limited thereto, but may be 0 bar or more, 50 bar or more, 100 bar or more, 150 bar or more, 200 bar or more, 250 bar or more, 300 bar or more, 350 bar or more, 400 bar or more, 450 bar or more, 500 bar or more, 550 bar or more, 600 bar or more, 650 bar or more, 700 bar or more, 750 bar or more, 800 bar or more, 850 bar or more, 900 bar or more, or 950 bar or more.

In addition, the pressure of the supercritical extraction is not limited thereto, but may be 1000 bar or less, 950 bar or less, 900 bar or less, 850 bar or less, 800 bar or less, 750 bar or less, 700 bar or less, 650 bar or less, 600 bar or less, 550 bar or less, 500 bar or less, 450 bar or less, 400 bar or less, 350 bar or less, 300 bar or less, 250 bar or less, 200 bar or less, 150 bar or less, 100 bar or less, or 50 bar or less.

The pressure conditions of the supercritical extraction are not limited to the range as long as it is possible to efficiently produce a nerve conduit by removing most of the cells of the nervous tissue while preserving the mechanical properties of the nervous tissue and the structural form of the tissue.

In the supercritical extraction step, a co-solvent may be further included in addition to the supercritical fluid. The co-solvent may be one or more solvents selected from the group consisting of ethanol, water, methanol, hexane, petroleum ether, acetonitrile, acetone, ethyl acetate, and methylene chloride. Preferably, ethanol may be further included as a co-solvent.

The co-solvent is added for the purpose of increasing the extraction ability and solubility of the supercritical fluid, extracting lipids in the separated nervous tissue, specifically phospholipids of the cell membrane, and removing most of the cells of the nervous tissue, and the type is not particularly limited as long as the mechanical properties of the nervous tissue and the structural form of the tissue are preserved.

In an embodiment of the present invention, carbon dioxide was used as a supercritical fluid when phospholipids were extracted from nervous tissue separated from pigs and the cells were removed. Since nonpolar carbon dioxide tends to be somewhat less efficient in extracting phospholipids that are both hydrophilic and lipophilic, ethanol, which is highly soluble in carbon dioxide and is hydrophilic, was used as a co-solvent to remove phospholipids.

In the present invention, the supercritical extraction is not limited thereto, but may be performed under temperature conditions of 31° C. to 40° C., 31° C. to 39° C., 32° C. to 38° C., 33° C. to 37° C., 34° C. to 36° C., or 35° C.

In the present invention, the supercritical extraction is not limited thereto, but may be performed for 60 minutes to 120 minutes, 70 minutes to 110 minutes, 80 minutes to 100 minutes, or 90 minutes.

The decellularized nerve conduit according to the present invention is not limited thereto, but may be produced including pretreating the nervous tissue with a hypertonic buffer before performing extraction with the supercritical fluid.

The hypertonic buffer may be pretreated on nervous tissue prior to supercritical fluid extraction for the purpose of increasing decellularization efficiency. Hypertonic buffers are well known in the art, and the hypertonic buffer is not limited thereto, but may be selected from the group consisting of Tris, sodium chloride (NaCl), glucose, mannitol, sodium bicarbonate, sodium acetate, lactate-containing saline and urea. At this time, the hypertonic buffer may preferably be Tris.

In addition, the hypertonic buffer may increase the decellularization efficiency of separated nervous tissue and shorten the overall decellularization process time, and the type is not particularly limited as long as the mechanical properties of the nervous tissue and the structural form of the tissue are preserved.

In an embodiment of the present invention, the efficiency of decellularization was increased by treating nervous tissue separated from pigs with Tris as a hypertonic buffer for 1 day before supercritical fluid extraction.

The decellularized nerve conduit according to the present invention, but is not limited thereto, may be produced including washing the nervous tissue with a phosphate buffer solution after the performing of the extraction with the supercritical fluid.

Through washing using the phosphate buffer, residual solution and impurities present in the nervous tissue after supercritical fluid extraction may be washed.

In an embodiment of the present invention, after supercritical fluid extraction, the decellularized nerve conduit was washed twice with phosphate buffered saline (PBS) to remove residual solution and impurities.

### Use of Decellularized Nerve Conduit

A nerve conduit according to the present invention is decellularized without surfactant treatment and maintains mechanical properties such as tensile strength and elasticity, making it easy for transplantation and suturing, and there is no problem of short circuiting again after connection surgery. In addition, transplant rejection was minimized through decellularization, and there was no toxicity due to residual surfactant. Therefore, the nerve conduit of the present invention obtained through an optimized supercritical fluid processing process is free of toxicity and transplant rejection, and thus may be useful for serious trauma, cubital tunnel syndrome, carpal tunnel syndrome, orthognathic surgery, or breast nerve regeneration.

### Method for Producing Decellularized Nerve Conduit

Another aspect of the present invention provides a production method of a decellularized nerve conduit, the production method including: a) pretreating nervous tissue separated from a subject with a hypertonic buffer; b) extracting the pretreated nervous tissue with supercritical fluid; and c) washing, with a phosphate buffer, the nervous tissue extracted with supercritical fluid.

The “hypertonic buffer,” “subject,” and “supercritical fluid” are as described above.

The a) step is pretreating the nervous tissue separated from the subject with a hypertonic buffer. Specifically, the decellularization efficiency of nervous tissue may be increased by pretreating the nervous tissue with a hypertonic buffer before supercritical fluid extraction. Additionally, the overall decellularization process time may be shortened.

As long as the hypertonic buffer preserves the mechanical properties of nervous tissue and the structural form of the tissue, the type is not particularly limited and may be, for example, selected from the group consisting of Tris, sodium chloride (NaCl), glucose, mannitol, sodium bicarbonate, sodium acetate, lactate-containing saline, and urea. Preferably, the hypertonic buffer may be Tris.

The b) step is extracting nervous tissue pretreated with a hypertonic buffer with a supercritical fluid, wherein lipid components in nervous tissue, specifically phospholipid components, which are the main components of cell membrane, may be decellularized by extracting them with supercritical fluid.

The type of supercritical fluid, pressure conditions, temperature conditions, and performance time for supercritical fluid extraction are as described in detail in “Production of decellularized nerve conduit”.

In addition, the supercritical fluid in the b) step is not limited thereto, but may further include a co-solvent. The co-solvent is added for the purpose of increasing the extractability and solubility of the supercritical fluid, and may extract lipids from the separated nervous tissue, specifically phospholipids of the cell membrane, and remove most of the cells of the nervous tissue.

As long as the co-solvent preserves the mechanical properties of nervous tissue and the structural form of the tissue, the type is not particularly limited and may be, for example, one or more solvents selected from the group consisting of ethanol, water, methanol, hexane, petroleum ether, acetonitrile, acetone, ethyl acetate, and methylene chloride. Preferably, ethanol may be further included as a co-solvent.

The c) step is washing the nervous tissue extracted with the supercritical fluid with a phosphate buffer, wherein by washing with the phosphate buffer, the residual solution and impurities present in the nervous tissue after extraction of the supercritical fluid may be washed.

### MODE FOR CARRYING OUT THE INVENTION

Hereinafter, the present invention will be described in more detail by the following examples. However, the following examples are only for illustrating the present invention, and the scope of the present invention is not limited to these only.

### Example 1. Production of Decellularized Nerve Conduit Using Supercritical Fluid Extraction Process

A supercritical fluid extraction process was performed to decellularize a nerve conduit.

First, nervous tissue was obtained from a pig, and muscle and fat tissue other than nervous tissue were removed. Afterwards, as a pretreatment process, the nervous tissue was treated with a hypertonic buffer (pH 8.0 Tris buffer) for 1 day. Nervous tissue treated with hypertonic buffer was put into an extraction tank of a supercritical extraction system (SES), and supercritical fluid carbon dioxide and co-solvent ethanol were injected into the extraction tank together. Afterwards, supercritical treatment was performed for 1 to 2 hours under pressure conditions of 200 bar to 400 bar and temperature conditions of 31° C. to 40° C. After supercritical treatment, a decellularized nerve conduit was finally obtained by performing washing twice with 1×PBS for 15 minutes.

### Comparative Example 1. Production of Decellularized Nerve Conduit Using Surfactant

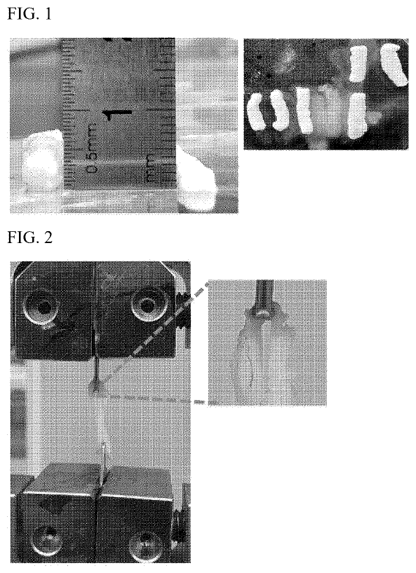

Decellularization of a nerve conduit using a surfactant was performed according to the Hudson method (see FIG. 1) described in a document, Mark Szynkaruk et. al., TISSUE ENGINEERING: Part B, 19 (1), 2013. At this time, the decellularized nerve conduit was produced using DOWFAX™ 2A1 solution (Dow Inc.) as a surfactant.

### I. Evaluation of Physicochemical Properties of Nerve Conduit According to Supercritical Fluid Extraction Process

### Experimental Example 1. Tensile Strength and Elasticity Testing of Decellularized Nerve Conduit Through Supercritical Fluid Extraction Process

The tensile strength and elasticity of decellularized pig nervous tissue prepared in Example 1 were tested. The tensile strength and elasticity of pig nervous tissue decellularized through a supercritical fluid extraction process were measured using a Universal Tensile Machine (EZ-x model (Shimadzu co.)). At this time, a thick sample with a size of 5 mm (D)×10 mm (L) and a thin sample with a size of 2.5 mm (D)×10 mm (L) were tested (FIG. 1). Test samples were an untreated group (N), a supercritical fluid treated group (SC), and a surfactant treated group (Detergent).

First, prior to the tensile strength test, decellularized pig nervous tissue was soaked in sterile distilled water to make a wet state. Both ends of the wet pig nervous tissue were hung on a universal tensile tester using a hook (FIG. 2). Afterwards, the tensile strength and Young's modulus were calculated by measuring the force when the tissue was broken by up and down pulling.

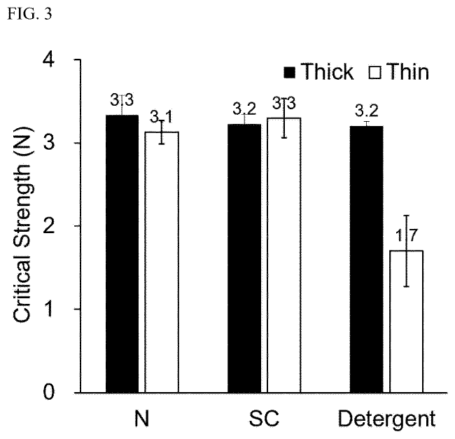

As a result, it was confirmed that the maximum tensile strength for thick and thin samples was 3 to 4 N for the untreated group and the supercritical fluid treated group. Meanwhile, in the case of the surfactant treatment group, the maximum tensile strength of the thin sample was confirmed to decrease to 1-2 N (45% reduction compared to the native tissue) (FIG. 3).

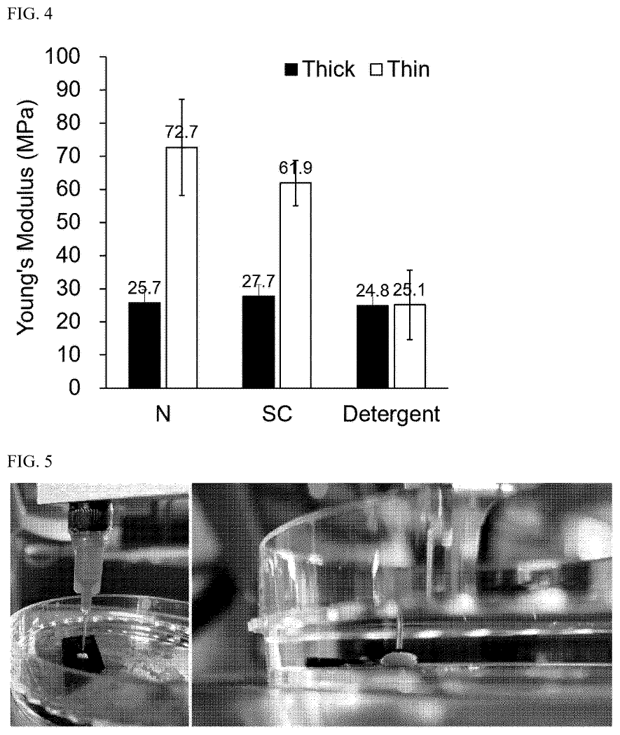

In addition, the Young's modulus of the thick sample was confirmed to be around 20 to 30 MPa for all the untreated group, supercritical fluid treated group, and surfactant treated group. The Young's modulus of the thin samples was confirmed to be as follows respectively (FIG. 4): the untreated group is at the 65 to 75 MPa level; the supercritical fluid-treated group is at the 65 to 75 MPa level (25% reduction compared to the native tissue); and the surfactant-treated group is at the 20 to 30 MPa level (65% reduction compared to the native tissue).

Through the above results, it was confirmed that in the case of thick nervous tissue, both the supercritical fluid treatment group and the surfactant treatment group were not affected in terms of changes in maximum tensile strength and Young's modulus compared to the native tissue. However, it was confirmed that the physical property preservation of nervous tissue decellularized using supercritical fluid for thin nervous tissue was 1.9 times and 2.5 times better than that of nervous tissue decellularized using surfactant in terms of maximum tensile strength and Young's modulus, respectively.

Therefore, when following the supercritical fluid treatment process according to the present invention, it was found that, compared to the case of decellularization using conventional surfactants, it is possible to provide an advantage in stably preserving physical properties regardless of the thickness and size of the donated nervous tissue.

### Experimental Example 2. Tissue Strength Measurement Test of Decellularized Nerve Conduit Through Supercritical Fluid Extraction Process

Tissue strength was measured for the decellularized pig nervous tissue prepared in Example 1. The tissue strength of the pig nervous tissue was measured using a UTM device (EZ-x model, Shimadzu co.) capable of measuring the physical properties of soft tissue and the degree of damage by repeated penetration tests. At this time, a blunt needle with a diameter of 0.8 mm was used as a probe for tissue penetration. Meanwhile, native pig nervous tissue that was not subjected to the supercritical fluid extraction process was used as a control, and decellularized pig nervous tissue obtained through the supercritical fluid extraction process (supercritical process) was used as a test group.

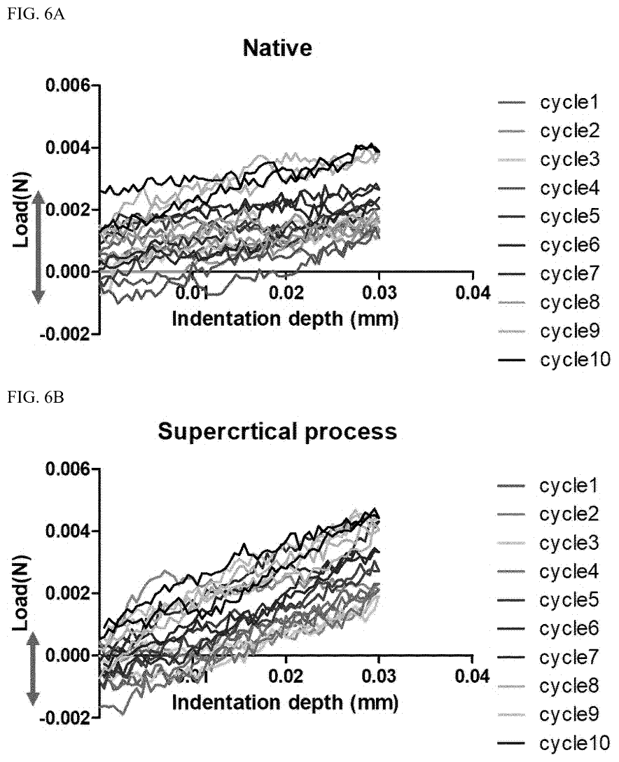

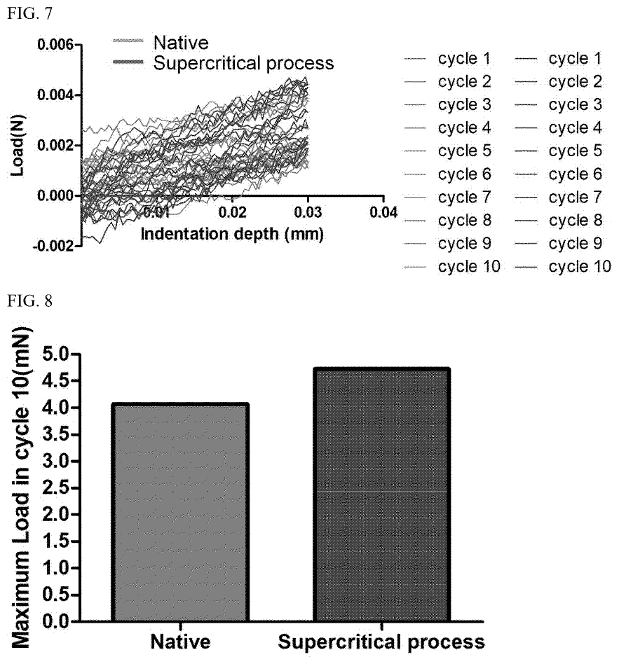

Specifically, all samples were hydrated and prepared before testing, and each tissue was fixed to the surface of a 60 pi dish using tape and silicone, as shown in FIG. 5. At this time, hydration was performed by sufficiently replenishing water on the dish 10 minutes before the test to absorb moisture. After hydrating the sample, the tester module was set to compression mode and a cyclic test was performed 10 cycles at a speed of 0.03 mm/min. At this time, the sample was repeated 6 times, and the load N value was measured (FIGS. 6A, 6B, and 7).

As a result, compared to native pig nervous tissue, it was confirmed that the tissue strength of decellularized pig nervous tissue obtained through a supercritical fluid extraction process increased from a maximum load of about 4 mN to about 5 mN without extreme damage in 10 cycles. That is, it was found that the tissue strength of the test group increased by about 10% compared to the control (FIG. 8).

### Experimental Example 3. Testing Resilience of Decellularized Nerve Conduit Through Supercritical Fluid Extraction Process

The resilience of the decellularized pig nervous tissue prepared in Example 1 was tested. The resilience of the pig nervous tissue was measured using a UTM device (EZ-x model, Shimadzu co.) capable of measuring the physical properties of soft tissue and the degree of damage by repeated penetration tests. At this time, a blunt needle with a diameter of 0.8 mm was used as a probe for tissue penetration. Meanwhile, native pig nervous tissue that was not subjected to the supercritical fluid extraction process was used as a control, and decellularized pig nervous tissue obtained through the supercritical fluid extraction process (supercritical process) was used as a test group.

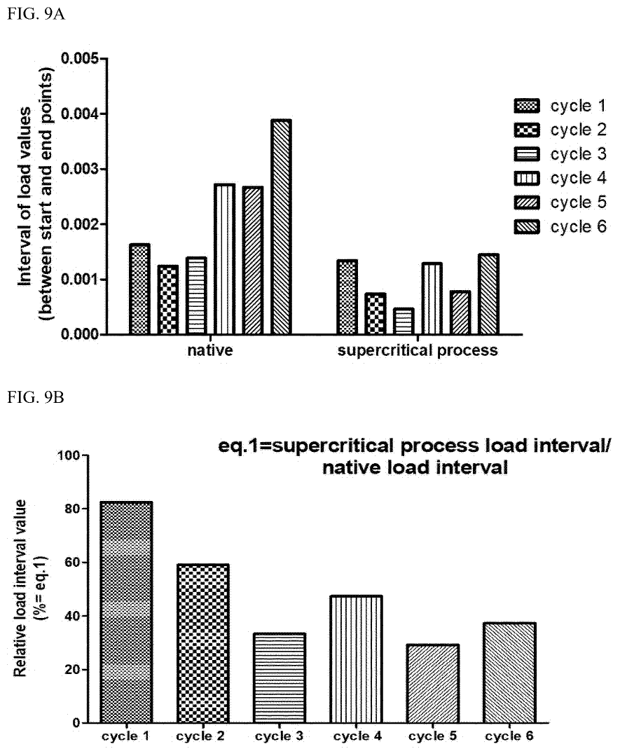

The resilience test was performed in the same manner as the tissue strength measurement test in Experimental Example 2, and the degree to which the tissue was deformed was measured at each repeated measurement cycle. That is, the resilience was tested as a measure to determine how much the elasticity of the tissue changes due to external forces generated during repeated compression tests. At this time, the test cycle was conducted 6 times, and the test was repeated 6 times on the sample. The test value for resilience was expressed by calculating the difference between the starting and ending points of the physical property measurement of the sample for each cycle (FIG. 9A). Meanwhile, using the derived difference value, the relative value between samples was calculated and shown for each cycle (FIG. 9B). The relative value was derived and expressed through a value that compares the degree of change in load value that occurs during the test between samples, eq. 1=[Load difference value of sample obtained through supercritical fluid extraction process/Load difference value of natural sample without supercritical fluid extraction]

As a result, it was confirmed that the ability to return to the starting point decreases as the number of tests increases in the case of natural pig nervous tissue samples due to the repeated vertical movement of the probe on the tissue surface. On the other hand, decellularized pig nervous tissue samples obtained through a supercritical fluid extraction process showed that this phenomenon was alleviated, confirming that the elasticity of the tissue increased. The resilience of natural pig nervous tissue was reduced by about 50%, and decellularized pig nervous tissue obtained through a supercritical fluid extraction process was confirmed to have no change in resilience (FIGS. 9A and 9B).

Through this, it was found that the elastic deformation of the decellularized nerve conduit obtained through the supercritical fluid extraction process was low, enabling stable nerve transplantation without suture rupture when used for damaged nerve suture surgery. Accordingly, it was ultimately found that the nerve conduit obtained through the supercritical fluid extraction process could be usefully used as a transplant material for the damaged peripheral nervous system.

### II. Evaluation of Biochemical Properties of Nerve Conduit According to Supercritical Fluid Extraction Process

### Experimental Example 4. Confirmation of DNA Content within Decellularized Nerve Conduit

To confirm the degree of decellularization of the decellularized pig nervous tissue prepared in Example 1, the DNA content in the decellularized nerve conduit was measured. At this time, native tissue without supercritical fluid treatment was used as a negative control, and nervous tissue treated with surfactant according to a conventional decellularization method was used as a positive control.

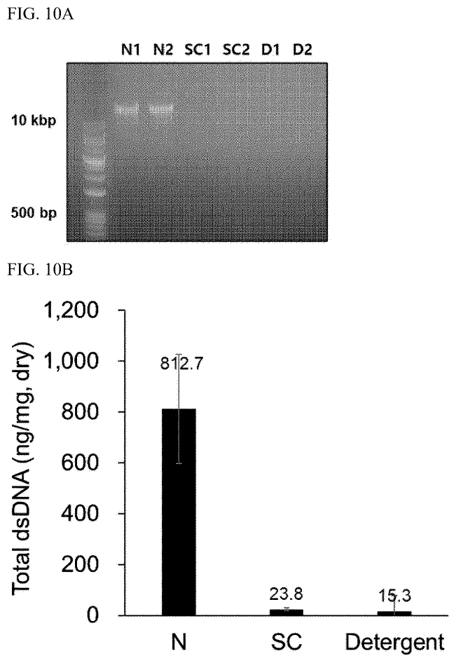

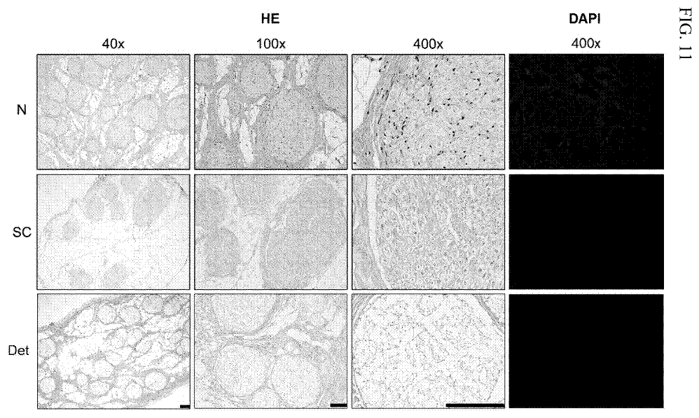

Specifically, each sample was lyophilized and completely dried, and then gDNA inside each nerve conduit was extracted using a DNA Extraction Kit (Intronbio). Afterwards, electrophoresis was performed using 1% agarose gel, and the DNA content inside the nerve conduit was confirmed by quantitative analysis (FIGS. 10A and 10B).

As a result of the experiment, the residual DNA content of both the pig nervous tissue decellularized according to the supercritical fluid process and the pig nervous tissue decellularized according to the conventional surfactant treatment process was confirmed to be less than 50 ng/mg based on dry weight.

Accordingly, it was found that the DNA removal effect during supercritical fluid treatment was equally excellent as in the conventional surfactant treatment process. In addition, as decellularization of more than 90% of the native tissue was confirmed, it was found to have excellent effects that can be used as a transplant material without transplant rejection.

### Experimental Example 5. Histological Analysis of Decellularized Nerve Conduit

For histological analysis of the decellularized pig nervous tissue prepared in Example 1, H&E staining and DAPI fluorescence staining were performed to analyze the histological form and degree of decellularization of the tissue. At this time, native tissue without supercritical fluid treatment was used as a negative control, and nervous tissue treated with surfactant according to a conventional decellularization method was used as a positive control.

As a result of the staining, it was confirmed that the histological form of the nerve conduit was maintained compared to the native tissue, while cell nuclei were removed to the same level as the nervous tissue treated with conventional surfactant (FIG. 11). That is, it was found that decellularization was effectively achieved.

### Experimental Example 6. Confirmation of Collagen Content in Decellularized Nerve Conduit

In order to confirm that there was no loss of protein in the decellularized pig nervous tissue prepared in Example 1, the collagen content was measured using the Sircol insoluble Collagen assay kit from Biocolar. Ltd. At this time, native tissue without supercritical fluid treatment was used as a negative control, and nervous tissue treated with surfactant according to a conventional decellularization method was used as a positive control.

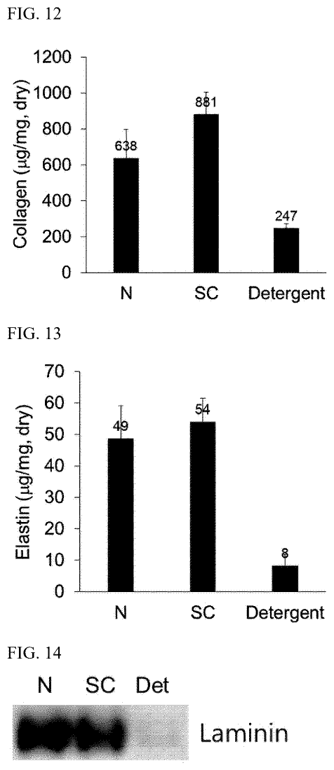

As a result of the measurement, the collagen content in pig nervous tissue decellularized according to a supercritical fluid process was confirmed to be 881 μg/mg based on the dry weight of a sample. This was at a level of 138% compared to the native tissue, showing that no protein loss occurred (FIG. 12).

On the other hand, in the case of nervous tissue treated with conventional surfactant, the collagen content was confirmed to be 247 μg/mg, which was only 39% of the native tissue (FIG. 12). That is, in the case of decellularization by conventional treatment with surfactant, it was found that although the decellularization effect was excellent, protein loss within the nerve conduit occurred significantly.

Through the results, it was found that the nerve conduit decellularized by supercritical fluid processing was effectively decellularized without protein loss while maintaining histological form and mechanical properties. Accordingly, it was found that there is an excellent effect that is usability as a transplant material.

### Experimental Example 7. Confirmation of Elastin Content in Decellularized Nerve Conduit

To confirm that there was no loss of elastin in the decellularized pig nervous tissue prepared in Example 1, elastin analysis was performed using the Fastin Elastin assay kit from Biocolar. Ltd. At this time, native tissue without supercritical fluid treatment was used as a negative control, and nervous tissue treated with surfactant according to a conventional decellularization method was used as a positive control.

As a result of the analysis, the elastin content in pig nervous tissue decellularized according to the supercritical fluid process was confirmed to be 54 μg/mg based on the dry weight of the sample. This was at a level of 110% compared to the native tissue, showing that no elastin loss occurred (FIG. 13).

On the other hand, in the case of nervous tissue treated with conventional surfactant, the elastin content was confirmed to be 8 μg/mg, which was only 16% of the native tissue (FIG. 13). That is, when decellularization was performed by conventional surfactant treatment, it was found that even though the decellularization effect was excellent, there was significant loss of elastin within the nerve conduit.

Through the results, it was found that the nerve conduit decellularized by supercritical fluid processing was decellularized without loss of elastin while maintaining histological form and mechanical properties, and had an excellent effect that is usability as a transplant material.

### Experimental Example 8. Confirmation of Laminin Preservation within Decellularized Nerve Conduit

To confirm the degree of laminin preservation in the decellularized pig nervous tissue prepared in Example 1, Western blot analysis was performed. At this time, native tissue without supercritical fluid treatment was used as a negative control, and nervous tissue treated with surfactant according to a conventional decellularization method was used as a positive control.

Specifically, Western blot was performed using a laminin antibody (Cat. No. NB600-883) from NOVUSBIO (FIG. 14). In addition, the Western blot analysis results were compared and analyzed by quantifying the band intensity using ImageJ (version 1.51j8) Software.

As a result of the analysis, the band intensity of pig nervous tissue decellularized according to the supercritical fluid process was derived to be 173. Meanwhile, the values were 190 for the native tissue and 58 for the nervous tissue treated with a surfactant. Converting this to a relative value compared to the native tissue, the laminin content in the nervous tissue decellularized according to the supercritical fluid process is 91%, and it was confirmed that the laminin content was preserved at a remarkable level compared to 31% in nervous tissue decellularized by conventional treatment with a surfactant.

### III. Confirmation of In Vivo Effects of Nerve Conduit According to Supercritical Fluid Extraction Process

The in vivo nerve regeneration effect was confirmed in a nerve defect rat model using the nerve conduit finally established through evaluation of the physicochemical and biochemical properties of the nerve conduit according to a supercritical fluid extraction process.

### Experimental Example 9. Confirmation of DNA Remaining within Decellularized Nerve Conduit

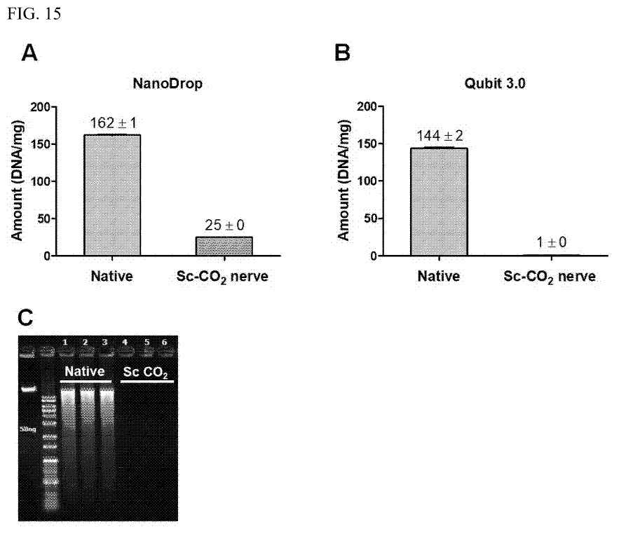

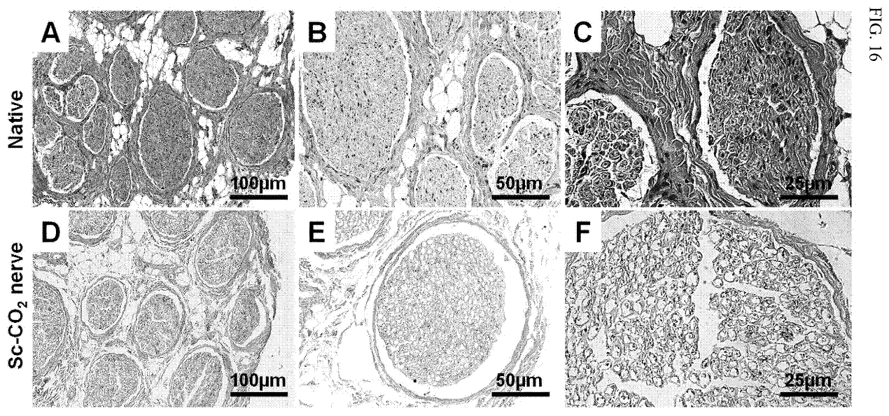

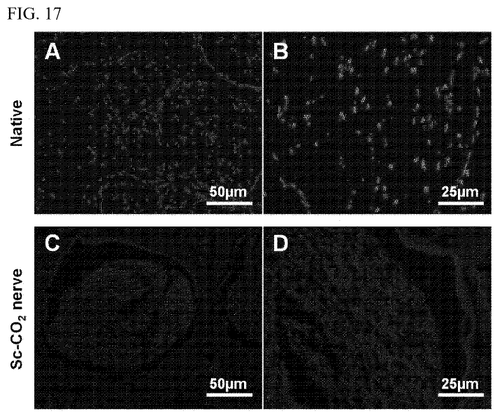

Prior to confirming the nerve regeneration effect in the nerve defect rat model, the residual DNA per mg was roughly quantified using nanodrops and qubits to determine whether DNA remained in the nerve conduit prepared as in Example 1 above. Additionally, the presence of DNA was confirmed through electrophoresis. As a result, dsDNA was at a significantly lower level in the nerve conduit (Sc-CO₂ nerve) decellularized by supercritical fluid treatment compared to the native tissue, and no DNA band was observed on the electrophoresis image, indicating that decellularization had been made (FIG. 15).

### Experimental Example 10. Confirmation of Histological Form and Degree of Decellularization of Decellularized Nerve Conduit

H&E staining and DAPI fluorescence staining were performed on a nerve conduit to be applied to a nerve defect rat model to analyze the tissue's histological form and degree of decellularization.

As a result of staining, it was confirmed that cell nuclei were removed while maintaining the histological form of the nerve conduit compared to the native tissue (FIGS. 16 and 17). That is, it was found that decellularization was effectively achieved.

### Experimental Example 11. Confirmation of Preservation of Extracellular Matrix (ECM) in Decellularized Nerve Conduit

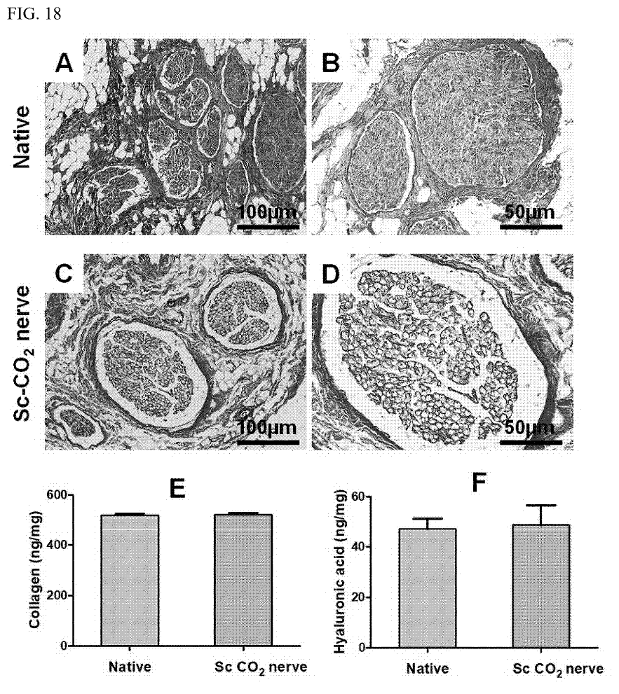

The nerve conduit to be applied to a nerve defect rat model was histologically confirmed for collagen preservation through Masson's Trichrome staining. In addition, collagen and hyaluronic acid (HA) were quantified using a commercially available Sircol insoluble Collagen assay kit (Biocolar, Cat. No. S2000) and a Hyaluronic acid ELISA kit (Biovision, Cat. no. E4626), respectively, so that it was confirmed whether an extracellular matrix of the decellularized nerve conduit was preserved. As a result, it was found that collagen and hyaluronic acid in pig nervous tissue decellularized according to the supercritical fluid process showed an excellent preservation rate at the same level without protein loss compared to the native tissue (FIG. 18).

### Experimental Example 12. Determination of Cytotoxicity of Decellularized Nerve Conduit

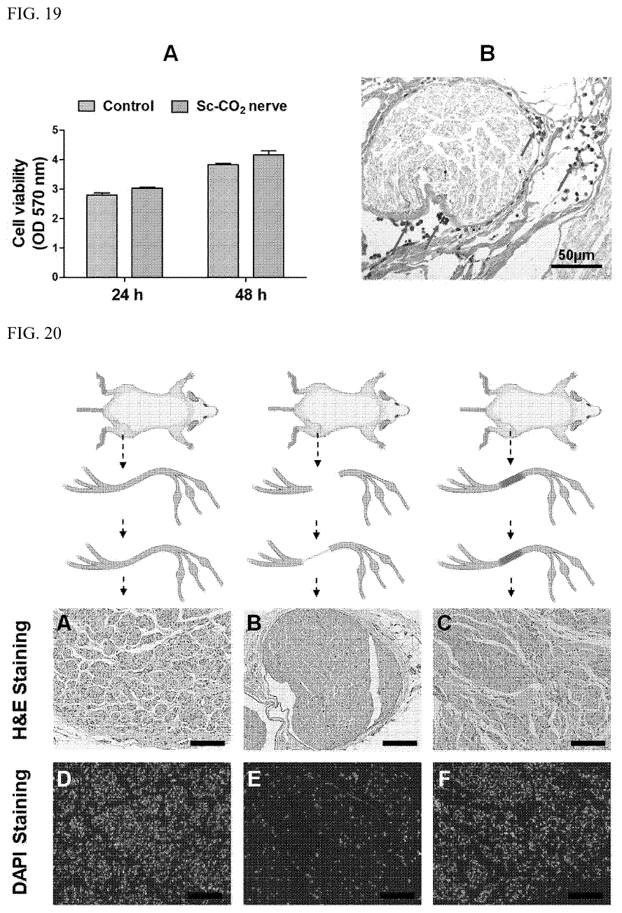

Fibroblasts were cultured on the decellularized nerve conduit tissue for 24 and 48 hours, respectively, and MTT assay was performed to confirm the degree of cytotoxicity of the decellularized nerve conduit. In addition, cytotoxicity was evaluated through migration into the decellularized nerve conduit tissue using fibroblasts of which nuclei were stained blue by H&E staining. As a result, it was confirmed that pig nervous tissue decellularized according to the supercritical fluid process maintained excellent cell survival rate compared to the native tissue (control). Additionally, migration of nuclear-stained fibroblasts into the nerve conduit tissue was observed (FIG. 19). Accordingly, it was found that the decellularized nerve conduit has no cytotoxicity and thus may be usefully used as a transplant material for damaged nervous tissue.

### Experimental Example 13. Confirmation of Nerve Regeneration Effect by Transplantation of Decellularized Nerve Conduit

The decellularized nerve conduit (Sc-CO₂ nerve) prepared in Example 1 was transplanted into a nerve defect rat model, and DAPI staining and H&E staining were performed 6 months later to confirm the nerve regeneration effect. At this time, a group in which 10 mm of the sciatic nerve was damaged and regenerated naturally without nerve conduit transplantation was used as a negative control, and a group in which no nerve defects or nerve conduit transplantation was performed was used as a normal control.

Specifically, in order to observe changes in the number of non-neural cells, peripheral nervous tissues of an Sc-CO₂ nerve transplantation experimental group (F in FIG. 20) and a negative control (E in FIG. 20) that regenerated naturally without a transplantation procedure after nerve cutting were stained with DAPI fluorescent dye and immunofluorescence analysis was performed. As a result, the Sc-CO₂ nerve transplant experimental group showed a significant increase in the number of cells in the nerve damaged area, and showed a similar level of cell number as the normal group (D in FIG. 20).

In the case of H&E (Hematoxylin-Eosin) staining for histological examination, rat nerve cells were observed growing inside the heterogeneous nerve graft in the Sc-CO₂ nerve transplant experimental group. In addition, nerve connections were regular and there were no signs of severe inflammation or fibrosis (C in FIG. 20).

As a result, it was confirmed through the effectiveness test of the nerve defect rat model that the nervous tissue of the experimental group transplanted with a decellularized nerve conduit was regenerated to the level of the normal group compared to the control that naturally regenerated after nerve cutting (FIG. 20). Therefore, excellent nerve regeneration effects were confirmed when transplanting decellularized nerve conduit through a supercritical fluid extraction process, and it was found that the decellularized nerve conduit may be used as a useful transplant material for repairing damaged nerves.

### Experimental Example 14. Confirmation of Schwann Cell Growth by Transplantation of Decellularized Nerve Conduit

The decellularized nerve conduit (Sc-CO₂ nerve) prepared in Example 1 was transplanted into a rat model of nerve defect, and after 6 months, the purity and density of Schwann's cells according to nerve regeneration were evaluated through immunocytochemical staining using $100 antibody (rabbit, 1:100, Dako, Denmark). At this time, the negative control and normal group were applied in the same manner as in Experimental Example 13 above.

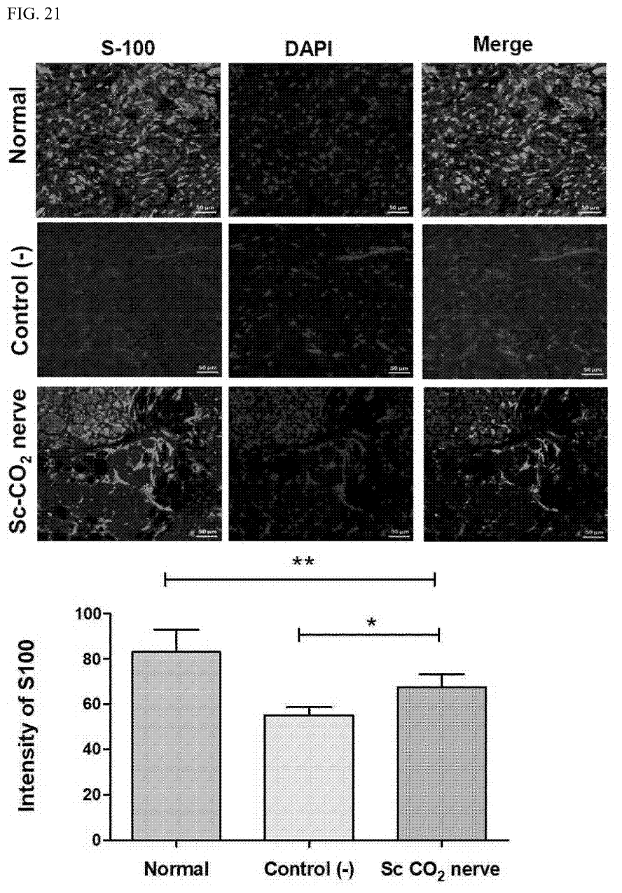

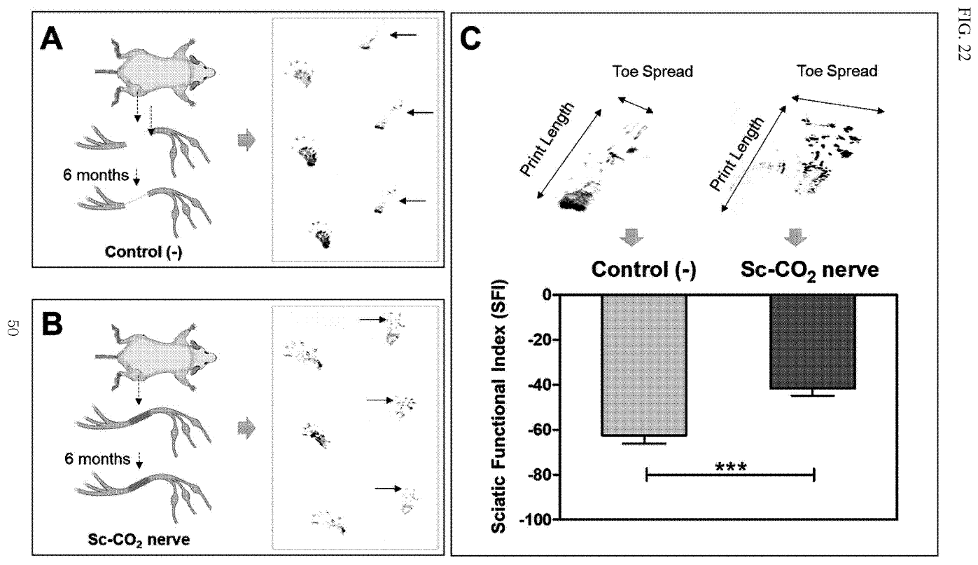

Schwann cells are peripheral nervous system cells with long bipolar or tripolar projections and a spindle-shaped cell shape with an oval nucleus, and as a result of analyzing the S100 protein as a marker by immunofluorescence, it was confirmed that the expression of the $100 Schwann cell marker protein increased in the Sc-CO₂ nerve transplant experimental group compared to the negative control (FIG. 21). That is, as shown in the merge image, there were more fluorescently stained Schwann cells in the nerves of the Sc-CO₂ nerve transplant experimental group compared to the negative control, showing that the nerve regeneration effect was excellent.

### Experimental Example 15. Evaluation of Motor Function Recovery in Animal Model of Nerve Defects by Decellularized Nerve Conduit Transplantation

A decellularized nerve conduit (Sc-CO₂ nerve) was transplanted into a rat model of nerve defect, and to evaluate the motor function recovery ability due to nerve regeneration in the nerve defect animal model after 6 months, walking track analysis was performed on all experimental animals (FIGS. 22A and 22B).

Specifically, ink was applied to the hind heels of rats and the rats were made to walk on a dark running path 80×100 cm long. Afterwards, the distance from the first toe to the tip of the fifth toe (Toe Spread, TS), the distance from the second toe to the tip of the fourth toe (Intermediate Toe spread, IT), and the distance from the heel to the tip of the third toe (Print length, PL) were confirmed. The measured values confirmed in this way were substituted into [Equation 1] below to calculate the sciatic nerve functional index (SFI). Based on the sciatic nerve function index calculated above, the degree of recovery of motor function in rats with nerve defects was quantitatively evaluated. At this time, the sciatic nerve function index is close to 0 in normal rats and reaches −100 in cases of complete damage.

$$S F I = - 38.3 ( \frac{EPL - NPL}{NPL} ) + 10 9.5 ( \frac{ETS - NTS}{NTS} ) + 1 3.3 ( \frac{EIT - NIT}{NIT} ) - 8.8 \qquad [ Equation 1 ]$$

In the above equation, PL (Print Length) is the distance from the heel to the third toe. TS (Toe Spread) is the distance from the first toe to the fifth toe. IT (Intermediate Toe Spread) is the distance from the second toe to the fourth toe. E (Experimental Leg) is the length of the foot of an experimental animal that underwent transplant surgery, and N (Nonexperimental Leg) is the length of the foot of a normal animal that did not undergo transplant surgery.

As a result of sciatic nerve function index analysis 6 months after transplantation, the SFI value was derived as −40.1 (n=5) in the Sc-CO₂ nerve transplant experimental group and −60 (n=5) in the negative control, and it was confirmed that there was a statistically significant difference between the two groups (C in FIG. 22). That is, the SFI value of the Sc-CO₂ nerve transplant experimental group was less close to −100 compared to the negative control, indicating that motor function recovery was superior to the control.

Therefore, through walking track analysis performed on a rat model of nerve defects, it was confirmed that transplantation of a decellularized nerve conduit had an excellent regenerative recovery effect in terms of motor functionality, and thus it was found that the decellularized nerve conduit is usable as a useful graft material for repairing damaged nerves.

1. A decellularized nerve conduit, wherein the nerve conduit, which is separated from a subject, has tensile strength of 2 to 5 N, Young's modulus of 20 to 80 Mpa, and elastic restoring force of 30 to 80% compared to native tissue.

2. The decellularized nerve conduit according to claim 1, wherein the nerve conduit has any one characteristic selected from the following group based on dry weight:
a DNA residual amount of 15 to 50 ng/mg; a collagen content of 300 to 1000 μg/mg; and
an elastin content of 10 to 60 μg/mg.

3. The decellularized nerve conduit according to claim 1, wherein the nerve conduit has any one characteristic selected from the following group compared to original group:
a DNA residual amount of 2 to 6%; a collagen content of 50 to 160%; an elastin content of 20 to 120%;
a laminin content of 40 to 95%; tensile strength of 90 to 110%;
Young's modulus of 80 to 110%; and
elastic restoring force of 30 to 80%.

4. The decellularized nerve conduit according to claim 1, wherein the nerve conduit, which is separated from a subject, is produced by a production method comprising performing extraction with supercritical fluid.

5. The decellularized nerve conduit according to claim 4, wherein a solvent used as the supercritical fluid is carbon dioxide.

6. The decellularized nerve conduit according to claim 4, wherein the supercritical fluid further comprises ethanol as a co-solvent.

7. The decellularized nerve conduit according to claim 4, wherein the extraction is performed under temperature conditions of 31° C. to 40° C.

8. The decellularized nerve conduit according to claim 4, wherein the extraction is performed under pressure conditions of 200 bar to 400 bar.

9. The decellularized nerve conduit according to claim 4, wherein the extraction is performed for 1 to 2 hours.

10. The decellularized nerve conduit according to claim 4, wherein the nervous tissue is pretreated with a hypertonic buffer before the performing of the extraction with the supercritical fluid.

11. The decellularized nerve conduit according to claim 4, wherein the nervous tissue is washed with a phosphate buffer solution after the performing of the extraction with the supercritical fluid.

12. A production method of a decellularized nerve conduit, the production method comprising:
a) pretreating nervous tissue separated from a subject with a hypertonic buffer;
b) extracting the pretreated nervous tissue with supercritical fluid; and
c) washing, with a phosphate buffer, the nervous tissue extracted with supercritical fluid.

13. The production method according to claim 12, wherein in the b), the supercritical fluid further comprises a co-solvent.
